(19) United States
(12) Patent Application Publication
(10) Pub. No.: US 2006/0224029 A1
(43) Pub. Date: Oct. 5, 2006

(54) Method of extending catalyst life in vinyl aromatic hydrocarbon formation

(75) Inventor: Jim Butler, League City, TX (US)

Correspondence Address: FINA TECHNOLOGY INC, PO BOX 674412, HOUSTON, TX, 77267-4412, US

(73) Assignee: FINA TECHNOLOGY, INC.

(21) Appl. No.: 11/092,491

(22) Filed: Mar. 29, 2005

(51) Int. Cl. C07C 2/64; C07C 4/06

(52) U.S. Cl. 585/444

(57) ABSTRACT

Methods of extending the life of dehydrogenation catalyst are described herein. For example, one embodiment includes providing a catalytic dehydrogenation system, wherein the catalytic dehydrogenation system includes at least one reaction vessel, the at least one reaction vessel loaded with a dehydrogenation catalyst including an alkali metal enhanced iron oxide, contacting the dehydrogenation catalyst with a feedstream including an alkyl aromatic hydrocarbon to form a vinyl aromatic hydrocarbon and contacting the feedstream with a catalyst life extender, wherein the catalyst life extender includes cesium.

### FIELD

Embodiments of the present invention generally relate to catalyst life extension in vinyl aromatic hydrocarbon formation.

### BACKGROUND

Catalytic dehydrogenation processes generally include the conversion of a paraffin alkylaromatic to the corresponding olefin in the presence of a dehydrogenation catalyst. During such dehydrogenation processes, it is desirable to maintain both high levels of conversion and high levels of selectivity. Unfortunately, dehydrogenation catalysts tend to lose activity when exposed to reaction environments, thereby reducing the level of conversion and/or the level of selectivity. Such losses may result in an undesirable loss of process efficiency. Various methods for catalyst regeneration exist, but such methods generally involve stopping the reaction process and in some cases, removing the catalyst for external regeneration, resulting in increased costs, such as costs related to heat loss and lost production.

Therefore, it is desirable to extend the life of such dehydrogenation catalysts without such increased costs.

### SUMMARY

Embodiments of the invention generally include a method of forming a vinyl aromatic hydrocarbon. The method generally includes providing a catalytic dehydrogenation system, wherein the catalytic dehydrogenation system includes at least one reaction vessel, the at least one reaction vessel loaded with a dehydrogenation catalyst including an alkali metal enhanced iron oxide, contacting the dehydrogenation catalyst with a feedstream including an alkyl aromatic hydrocarbon to form a vinyl aromatic hydrocarbon and contacting the feedstream with a catalyst life extender, wherein the catalyst life extender includes cesium.

Another embodiment generally includes a catalytic dehydrogenation system. The system generally includes at least one reaction vessel, the at least one reaction vessel loaded with a dehydrogenation catalyst including an alkali metal enhanced iron oxide. The at least one reaction vessel includes a vessel inlet adapted to provide a feedstream to the dehydrogenation catalyst and a vessel outlet adapted to pass a vinyl aromatic hydrocarbon therethrough. The system further includes a supply system adapted to provide a catalyst life extender to the feedstream, wherein the catalyst life extender includes cesium.

### BRIEF DESCRIPTION OF DRAWINGS

FIG. 1 illustrates a catalytic dehydrogenation system.

FIG. 2 illustrates a multistage catalytic dehydrogenation system.

### DETAILED DESCRIPTION

Introduction and Definitions

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions, when the information in this patent is combined with available information and technology. Various terms as used herein are shown below. To the extent a term used in a claim is not defined below, it should be given the broadest definition persons in the pertinent art have given that term as reflected in printed publications and issued patents at the time of filing.

As used herein, the term “conversion” means the percentage of paraffins or alkylaromatic hydrocarbon transformed.

The term “selectivity” means percentage of alkylaromatic hydrocarbon transformed to the desired product.

The term “activity” refers to the weight of product produced per weight of the catalyst used in the dehydrogenation process per hour of reaction at a standard set of conditions (e.g., grams product/gram catalyst/hr).

The term “loaded” refers to introduction of a catalyst within a reaction vessel.

As used herein, the term “alkali metal” includes but is not limited to, potassium, sodium, lithium and other members of the group IA and IIA metals of the periodic table, such as rubidium and cesium.

As used herein, the term “regeneration” means a process for renewing catalyst activity and/or making the catalyst reusable after it's activity has reached an unacceptable level. Examples of such regeneration may include passing steam over the catalyst bed or burning off carbon residue.

Process

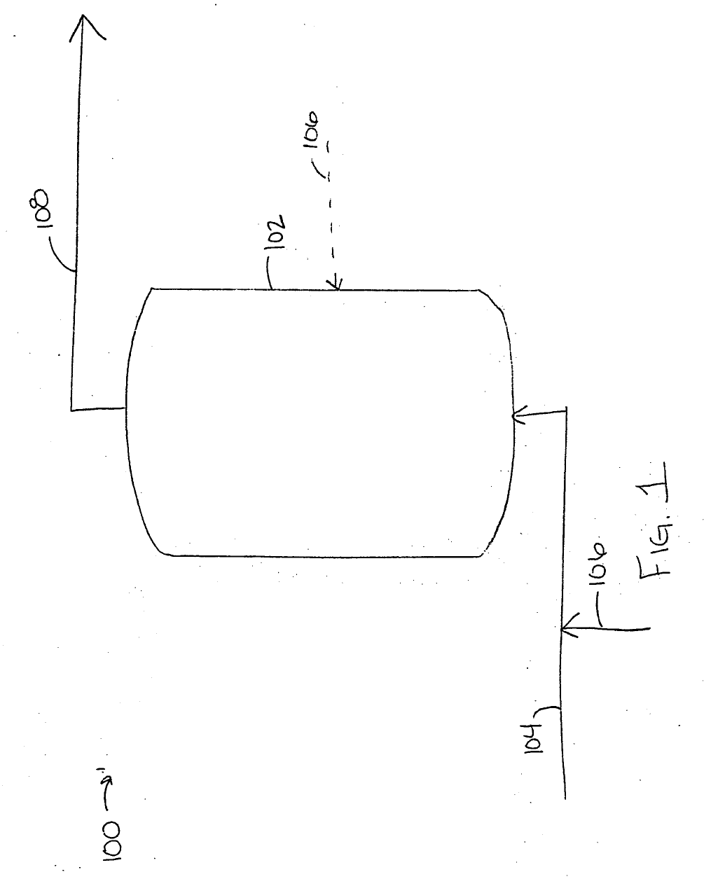

FIG. 1 illustrates a catalytic dehydrogenation system 100 including at least one reaction vessel 102 loaded with a dehydrogenation catalyst (not shown). An alkyl aromatic hydrocarbon (AAH) feedstream 104 enters the reaction vessel 102 and contacts the dehydrogenation catalyst to form a vinyl aromatic hydrocarbon (VAH) exit stream 108. Although the process is described here in terms of an alkyl aromatic hydrocarbon feedstream and a vinyl aromatic hydrocarbon exit stream, it is within embodiments of the invention described herein that the feedstream may be and/or include other compounds that may be contacted with a dehydrogenation catalyst to form a product, such as propane (converted to propylene) or butylene (converted to butadiene.)

One example of a catalytic dehydrogenation process includes dehydrogenating alkyl aromatic hydrocarbons over a solid catalyst component in the presence of steam (not shown) to form the VAH. Generally, the steam contacts the AAH feedstream 104 prior to the AAH feedstream 104 entering the reaction vessel 102, but may be added to the system 100 in any manner known to one skilled in the art. Although the amount of steam contacting the AAH is determined by individual process parameters, the AAH feedstream 104 may have a steam to AAH weight of from about 0.01 to about 15:1, or from about 0.3:1 to about 10:1, or from about 0.6:1 to about 3:1, or from about 0.8:1 to about 2:1, for example.

One specific embodiment includes the conversion of ethylbenzene to styrene, where the VAH exit stream 108 may include styrene, toluene, benzene, and/or unreacted ethylbenzene, for example. In other embodiments, the process includes the conversion of ethyltoluene to vinyltoluene, cumene to alpha-methylstyrene and/or normal butylenes to butadiene, for example.

The dehydrogenation processes discussed herein are high temperature processes. As used herein, the term “high temperature” refers to process operation temperatures, such as reaction vessel and/or process line temperatures (e.g., the temperature of the feedstream at the vessel inlet) of from about 150° C. to about 1000° C., or from about 300° C. to about 800° C., or from about 500° C. to about 700° C., or from about 550° C. to about 650° C., for example.

A variety of catalysts can be used in the catalytic dehydrogenation system 100. A representative discussion of some of those catalysts (e.g., dehydrogenation catalysts) is included below, but is in no way limiting the catalysts that can be used in the embodiments described herein.

The dehydrogenation catalysts discussed herein generally include an iron compound and at least one alkali metal compound. For example, the dehydrogenation catalyst may include from about 40 weight percent to about 90 weight percent iron, or from about 70 wt. % to about 90 wt. % iron, or from about 80 wt. % to about 90 wt. % iron. The iron compound can be iron oxide, or another iron compound known to one skilled in the art.

Further, the dehydrogenation catalyst may include from about 5 weight percent to about 60 weight percent alkali metal compound, or from about 8 wt. % to about 30 wt. % alkali metal compound, for example. The alkali metal compound may be potassium oxide, potassium hydroxide, potassium acetate, potassium carbonate or another alkali metal compound known to one skilled in the art, for example.

In another embodiment, the alkali metal compound may include cesium rather than potassium, such as cesium hydroxide, cesium acetate or cesium carbonate, for example. Although potassium is generally used for the dehydrogenation catalyst for numerous reasons, including cost, it has been found that cesium based catalysts may actually provide an activity similar to that of potassium based catalysts, while retaining adequate selectivity. See, Emersion H. Lee, Catalysis Reviews, 8(2), 285-305(1973).

Additionally, the dehydrogenation catalysts may further include additional catalysis promoters (e.g., up to about 20 wt. % measured as their oxides, or from about 1 wt. % to about 4 wt. %), such as nonoxidation catalytic compounds of Groups IA, IB, IIA, IB, IIIA, VB, VIB, VIIB and VIII and rare earth metals, such as zinc oxide, magnesium oxide, chromium or copper salts, potassium oxide, potassium carbonate, oxides of chromium, manganese, aluminum, vanadium, magnesium, thorium and/or molybdenum, for example.

Such dehydrogenation catalysts are well known in the art and some of those that are available commercially include: the S6-20, S6-21 and S6-30 series from BASF Corporation; the C-105, C-015, C-025, C-035, and the FLEXICAT series from CRI Catalyst Company, L.P.; and the G-64, G-84 and STYROMAX series from Sud Chemie, Inc. Dehydrogenation catalysts are further described in U.S. Pat. No. 5,503,163 (Chu); U.S. Pat. No. 5,689,023 (Hamilton, Jr.) and U.S. Pat. No. 6,184,174 (Rubini, et al.), which are incorporated by reference herein.

The dehydrogenation catalyst may be loaded into any reaction vessel 102 known to one skilled in the art for the conversion of an AAH to a VAH. For example, the reaction vessel 102 may be a fixed bed vessel, a fluidized bed vessel and/or a tubular reactor.

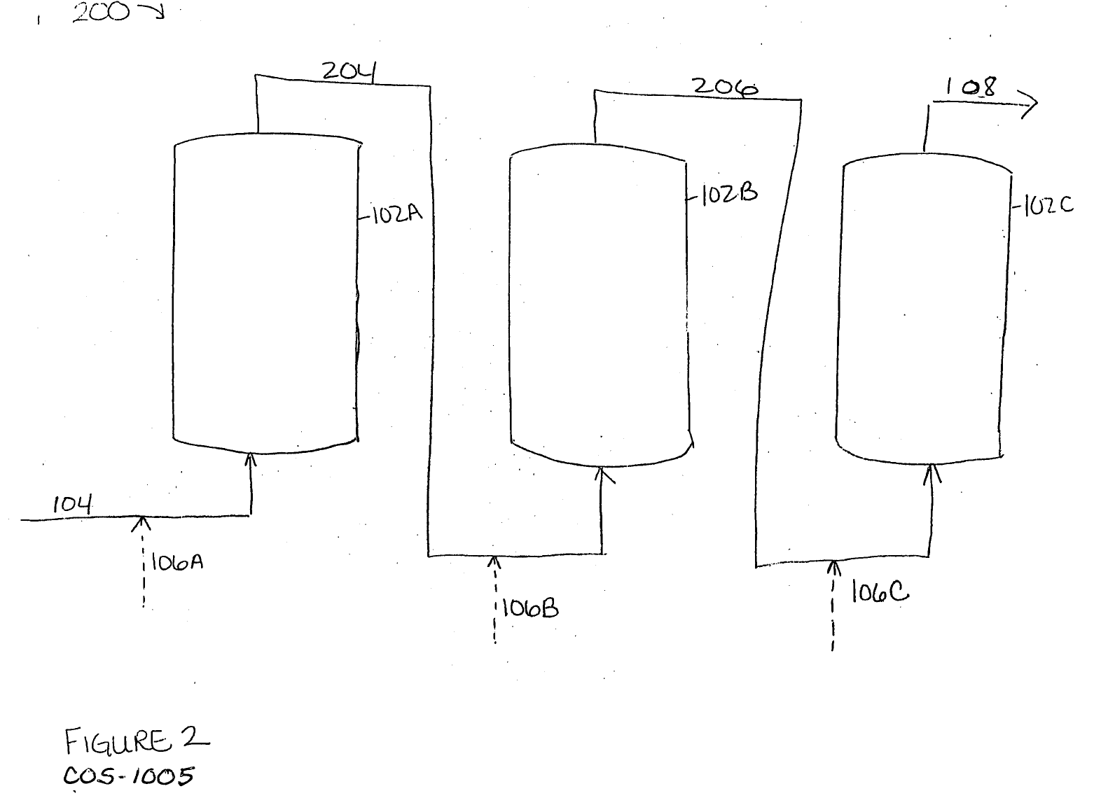

Although a single stage process is shown in FIG. 1, multistage processes are often utilized to form vinyl aromatic hydrocarbons and an example of such (three stages 200) is shown in FIG. 2. Although FIG. 2 illustrates three reactors/stages, any number or combination of reactors may be utilized. In a multistage process, such as process 200, the exit stream (204, 206) of one reaction vessel (102A, 102B) becomes the feedstream (204, 206) to another reaction vessel (102B, 102C). Therefore, when the dehydrogenation process is a multistage process, the term “feedstream” as used herein, may be the exit stream from a previous reactor, a “fresh” feedstream and/or a recycled stream, for example. In such embodiments, the feedstream (e.g., 204, 206) may include steam, partially reacted alkyl aromatic hydrocarbon, unreacted alkyl aromatic hydrocarbon and/or vinyl aromatic hydrocarbon, for example. Further, it is known in the art that additional process equipment, such as reheaters (not shown) may be included to maintain and/or restore process stream temperatures within a desired range, such as within a high temperature range at a reaction vessel inlet.

One process for preparing vinyl aromatic hydrocarbons is the “Dow Process”, which supplies superheated steam (720° C.) to a vertically mounted fixed bed catalytic reactor. The steam is generally injected into the reactor in the presence of a vaporized feedstream. See, The Chemical Engineers Resource Page at www.cheresources.com/polystymonzz.shtml.

Catalyst Life Extender

During such dehydrogenation processes, it is desirable to maintain both high levels of conversion and high levels of selectivity. Unfortunately, catalysts tend to lose activity when exposed to reaction environments, thereby reducing the level of conversion and/or the level of selectivity. Such losses may result in an undesirable loss of process efficiency. Various methods for catalyst regeneration exist, but such methods generally involve stopping the reaction process and in some cases, removing the catalyst for external regeneration, resulting in increased costs, such as costs related to heat loss and lost production.

One method for overcoming the loss of catalyst activity includes raising the temperature of the feedstream and/or the reaction vessel. Such temperature increases raise the rate of reaction in order to offset the continuing loss of catalyst activity. The embodiments described herein contemplate such temperature increases in combination with other processes for catalyst regeneration. Unfortunately, above a certain temperature, the mechanical temperature limit of the process equipment or the dehydrogenation catalyst may be reached, thereby increasing the potential degradation of the catalyst physical structure and/or the integrity of the process equipment.

Returning to FIG. 1, one regeneration method that is described further below includes the addition of a catalyst life extender (CLE) 106 to the dehydrogenation process 100. The CLE 106 may be added to the system 100 at various points, including the reaction vessel 102, the catalyst bed (not shown) and/or process stream 104, for example. Such processes may avoid/delay the need for catalyst removal from the reaction vessel 102 for regeneration and/or disposal.

The catalyst life extender 106 may be selected from non-halogen sources of alkali metal ions and may include a combination thereof. The amount of catalyst life extender 106 added to the process depends at least in part on the reaction conditions, equipment, feedstream composition and/or the catalyst life extender 106 being used, for example.

Such catalyst life extenders 106 may include potassium based compounds, such as potassium hydroxide. Unfortunately, addition of potassium hydroxide generally results in costly addition methods, such as the vaporization of molten potassium in order to eliminate and/or reduce fouling. For example, in the initial phases of industry implementation, aqueous potassium hydroxide (KOH) addition was attempted. It was determined that KOH addition, with the KOH being at ambient temperature, resulted in severe reactor fouling and plugging of the injection hardware and/or process line. Such fouling may be the result of potassium hydroxide's high melting point, resulting in solids formation and deposit. Therefore, KOH catalyst life extenders are generally preheated to a temperature similar to that of the feedstream prior to addition.

However, in one embodiment, the catalyst life extender 106 is a compound containing potassium, is neither excessively deliquescent nor dangerously reactive and has a melting point or vapor point such that it can be used at dehydrogenation process temperatures without blocking process lines or fouling process equipment. For example, the catalyst life extender 106 may be a potassium salt of a carboxylic acid, such as potassium acetate.

Unexpectedly, it has been found that such catalyst life extenders (in aqueous form) are capable of being injected into high temperature process lines without the expected plugging/fouling. Rather, aqueous addition of the carboxylic acids described above resulted in markedly decreased fouling and in some instances, no fouling for extended periods of time. Previous attempts at aqueous potassium hydroxide addition resulted in plugging/fouling after only a short period of time, such as days, versus weeks or months.

In another embodiment, the catalyst life extender 106 is a compound containing cesium, such as cesium hydroxide, for example. Unlike potassium hydroxide, cesium hydroxide has a melting point of about 272° C. and would therefore vaporize into the steam. Further, the decomposition temperature of cesium carbonate is about 610° C., which would likely result in little if any formation of cesium carbonate byproducts, which may foul the reactor and/or process lines. Therefore, cesium based catalyst life extenders provide for aqueous catalyst life extender injection into the feedstream, while reducing, if not eliminating reactor and process line fouling due to such injection.

Further, the catalyst life extender 106 is generally substantially free of any catalysts poisons. For example, it has been reported that halogen ions, such as chloride, may poison dehydrogenation catalysts. Therefore, the catalyst life extender 106 includes little or no halogen substituents.

The catalyst life extender 106 may be supplied to the system 100 at a rate equivalent to a continuous addition of from about 0.01 to about 100 parts per million by weight of catalyst life extender relative to the weight of the total alkyl aromatic hydrocarbon in the feedstream 104, or from about 0.10 to about 200 parts per million, for example.

Just as the catalysts life extenders can be introduced into the dehydrogenation process by more that one method, it is also within the scope of the present invention to introduce the catalyst life extenders 106 to the dehydrogenation process at more than one rate. For example, the catalyst life extenders 106 can be introduced continuously or periodically, such as when catalyst activity levels fall below a predetermined level. In still another embodiment, the catalyst life extenders may be added at a relatively low level with additional catalyst life extender being added to the process when catalyst activity levels fall below a predetermined level. Accordingly, the system may include monitoring means (not shown) to monitor temperatures and chemical compositions to determine when conversion drops below a predetermined level.

### EXAMPLE

A steam and ethylbenzene feedstream was contacted with a potassium enhanced iron oxide dehydrogenation catalyst in a reaction to form styrene. The feedstream (10:1 molar ratio of steam:ethylbenzene) was fed to the reaction at a temperature of about 1200° F. (649° C.) via a conduit. Prior to the reactor inlet, aqueous potassium acetate was injected into the first conduit to contact and mix with the feed stream. The potassium acetate was at ambient temperature prior to injection.

Two months after startup of the above process, a gamma scan of the conduit and the reactor observed essentially no deposits therein.

What is claimed is:

1. A method of forming a vinyl aromatic hydrocarbon comprising:
providing a catalytic dehydrogenation system, wherein the catalytic dehydrogenation system comprises at least one reaction vessel, the at least one reaction vessel loaded with a dehydrogenation catalyst comprising an alkali metal enhanced iron oxide;
contacting the dehydrogenation catalyst with a feedstream comprising an alkyl aromatic hydrocarbon to form a vinyl aromatic hydrocarbon; and
contacting the feedstream with a catalyst life extender, wherein the catalyst life extender comprises cesium.

2. The method of claim 1, wherein the alkyl aromatic hydrocarbon comprises ethylbenzene and the vinyl aromatic hydrocarbon comprises styrene.

3. The method of claim 1, wherein the catalytic dehydrogenation system is a multistage process.

4. The method of claim 1, wherein the catalyst life extender comprises cesium hydroxide, cesium carbonate or combinations thereof.

5. The method of claim 1, wherein the catalyst life extender contacts the feedstream at a rate equivalent to a continuous addition of from about 0.01 ppm to about 100 ppm by weight of catalyst life extender relative to the weight of the alkyl aromatic hydrocarbon.

6. The method of claim 1, wherein the catalyst life extender contacts the feedstream during the formation of the vinyl aromatic hydrocarbon.

7. The method of claim 1, wherein the feedstream further comprises steam.

8. A catalytic dehydrogenation system comprising:
at least one reaction vessel, the at least one reaction vessel loaded with a dehydrogenation catalyst comprising an alkali metal enhanced iron oxide and wherein the at least one reaction vessel comprises a vessel inlet adapted to provide a feedstream comprising an alkyl aromatic hydrocarbon to the dehydrogenation catalyst and a vessel outlet adapted to pass a vinyl aromatic hydrocarbon therethrough; and
a supply system adapted to provide a catalyst life extender to the feedstream, wherein the catalyst life extender comprises cesium.
